(19) United States
(12) Patent Application Publication
(10) Pub. No.: US 2003/0111545 A1
(43) Pub. Date: Jun. 19, 2003

(54) Air freshener device

(76) Inventor: Anthony E. Girone, Toms River, NJ (US)

Correspondence Address: John O. Mingle, P.O. Box 1311, Laramie, WY, 82073-1311, US

(21) Appl. No.: 10/350,571

(22) Filed: Jan. 24, 2003

Related U.S. Application Data
(63) Continuation of application No. 10/228,327, filed on Aug. 26, 2002, now abandoned.
(60) Provisional application No. 60/314,550, filed on Aug. 24, 2001.

(51) Int. Cl. A61L 9/04

(52) U.S. Cl. 239/57; 239/34; 239/53; 239/211

(57) ABSTRACT

An air freshener device designed to be efficient, simple and inexpensive that is utilized in many convenient ways and allows construction in a wide variety of conventional shapes and sizes, while at the same time being reusable with different scents by just washing and recharging.

### BACKGROUND OF INVENTION

This application is a continuation of application Ser. No. 10/228,327, filed 08/26/02, now abandoned, which is itself a continuation of Provisional Application Serial No. 60/314,550, filed Aug. 24, 2001.

1. Field of Invention

The present invention relates to an apparatus used as an air freshener and the manner in which it can be constructed

2. Background

Air fresheners are a common item and have been with our society for scores of years. Many designs and types exist and they have in common a method of passing air over a substance that is giving off a given air freshener vapor, such as potpourri. These air fresheners are made in various shapes and positioned at various places around the home, garage and car.

Many of these air fresheners have elaborate procedures to pass enough air over the freshening substance to produce the desired effect. Conversely the subject invention primarily employs material that air passes through readily by natural means. Many of these air fresheners are not reusable and are thrown away after their ability to continue to be an adequate air freshener expires. Conversely the subject invention is reusable either by recharging with the same freshening substance, or if a different perfume is utilized, just wash it before the new application.

Related United States patents include:

| No. | Inventor | Year |
|---|---|---|
| 6,254,836 B1 | Fry | 2001 |
| Des. 426,294 | Tradeau et al. | 2000 |
| Des. 390,941 | Cessaroni et al. | 1998 |
| 5,788,061 | Hammond | 1998 |
| 5,823,432 | Hogan | 1998 |
| 5,503,332 | Glenn | 1996 |
| 5,383,598 | Styles | 1995 |
| 1,960,149 | Gilot | 1934 |

Fry discloses an air freshener that employs an internal bulb to force air over liquid scented oil before it leaves through selected apertures. This special dispensing apparatus is situated within a much larger body that is identified with artistic native American shapes.

Tradeau et al. in their design patent disclose only an artistic scent sack configuration with no indication of materials, sizes, or operation.

Cessaroni et al. in their design patent disclose only an elaborate and artistic flower shaped air freshener with no indication of materials, sizes, or operation.

Hammond discloses a two compartment potpourri container in the shape of an envelope that can be either be flat for shipping or popped up to a A-frame configuration for use and composed of paper or breathable plastic.

Hogan discloses an air freshener device as a form that assumes the shape of an article of clothing into which it is inserted The form holds an air permeable pouch to receive scented beads.

Glenn discloses a scent packet composed of a wick sheet containing perfume oil that is wrapped by a plastic film, and then placed with a plastic bag, which itself is inserted into a paper envelope. The inner layers are designed to slow up the release of the scent thus producing a long lasting air freshener.

Styles discloses an air freshener retainer hung with a strap and made of matched opposing sides fastened together to form a pocket for scented material, and further composed of material not allowing the scented material to contaminate contacted surfaces. Separate apertures are provided for needed air circulation.

Gilot discloses foldable paper container surrounding a bag holding scented material and formed so that by unfolding the container air aperture holes are exposed allowing the vapor to escape.

### SUMMARY OF INVENTION

The objectives of the present invention include overcoming the above-mentioned deficiencies in the prior art by designing an air freshener device that is simple, reusable and flexible in its positioning while being relatively inexpensive.

### BRIEF DESCRIPTION OF DRAWINGS

FIG. 1 shows a front view of a typical air freshener in the design of a mystical face, while FIG. 2 shows its rear view.

### DETAILED DESCRIPTION OF INVENTION

The subject invention is a simple air freshener device allowing it to be utilized in any convenient manner and allowing construction in a wide variety of shapes and sizes, while at the same time being reusable with different scents by just washing and recharging.

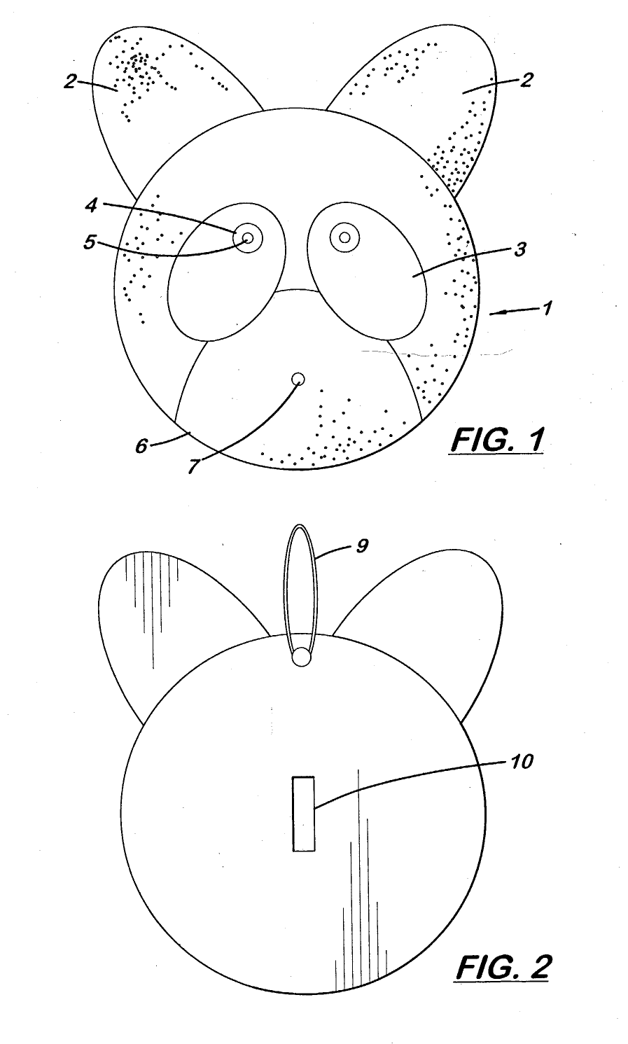

FIG. 1 shows a typical configuration of the subject invention where a carpet piece for the back side 1 of circular shape is employed to form a basic mystical face by adding ears 2 fixed in a similar manner from partial circles. The mystical face is then composed of various circles and ellipses of material, such as felt or fake-fur that is air passing, for the inner eyes 4 and outer eyes 5 positioned on top of cheeks 3 with the mouth 7 and chin 6 all composed similarly by gluing at the edges onto the back side 1. This composite now forms an inner chamber that can hold the scented potpourri, oil, or other form of perfume.

FIG. 2 shows the rear of the mystical face with its attached hanger string 9 and magnet 10 for positioning on a magnetic attracting metal, such as a refrigerator door. Also on the rear is a slit aperture for charging the chamber, and in FIG. 2 this is not shown directly but exists behind the magnet 10 that can become removable by Velcro usage. Alternatively this slit aperture in to chamber can be positioned in a different location.

This simple design is all washable so that if the kind of perfume is to be changed, the air freshener is just washed and let dry before recharging with a different type of scent oil.

The simplicity of such design keeps the cost low and at the same time provides a wide variety of shapes available for marketing such air fresheners. The shape of the air freshener shown in FIGS. 1 and 2 is for explanatory purposes only and does not restrict the shape of additional air fresheners produced under the subject invention.

An apparatus for an air freshener comprising a body having a front side material allowing air ventilation and a rear side material, wherein said front side is further made into a recognizable or identifiable shape, and such shape is potentially both from mystical origins as well as natural occurring forms. Further the front side material is composed of one or more individual pieces. The front side and rear side are connected at overlying edges by employing glue, thread, clips, fasteners, staples, tapes, Velcro-type tapes, and combinations thereof, and this forms an inner chamber capable of holding chemically treated aromatically emissive substances. A slit opening is made into said chamber capable of positioning said chemically treated aromatically emissive substances. Finally said body is potentially connected or attached to an external fixed location by employing hooks, strings, rubber bands, magnets, tape, Velcro-type tapes, staples, clips, fasteners, and combinations thereof, yet, it is still employable without such attachment.

The chemically treated aromatically emissive substances are selected from the group consisting of potpourri, perfumed oils, natural perfumes, natural biological liquids, and combinations thereof These generally are expected to be of a perfume-type odor; however, other non-perfume types of odor are possible.

The material employed is selected from the group consisting of carpet, fake-fur, felt, filter base, woven cloth, and combinations thereof. The front side material is air passing when it covers the chamber. The rear side material may or may not be capable of air ventilation. All material employed is washable.

The recognizable shape is selected from the group consisting of circles, squares, triangles, rectangles, ellipses, hearts, animal faces, animal bodies, birds, biological vegetation, and combinations thereof, that can be combined to produce both natural and mystical shapes. This further allows the making of various faces, bodies, trees, and other generally identifiable shapes, which are generally unartistic. Here “unartistic” means a design for the shape that is largely composed of conventional geometric and natural type components rather than drawn in a free-hand sense with elaborate aesthetic representation.

The foregoing description of the specific embodiments will so fully reveal the general nature of the invention that others can, by applying current knowledge, readily modify and/or adapt for various applications such specific embodiments without departing from generic concept, and therefore such adaptations or modifications are intended to be comprehended within the meaning and range of equivalents of the disclosed embodiments. It is to be understood that the phraseology or terminology herein is for the purpose of description and not of limitation.

What is claimed is:

1. An apparatus for an air freshener comprising:
a body having a front side material allowing air ventilation and a rear side material, wherein said front side is further made into an identifiable shape;
means for attaching together said front side and rear side at overlying edges forming at least one inner chamber capable of holding chemically treated aromatically emissive substances; and
a slit opening into said chamber capable of positioning said chemically treated aromatically emissive substances.

2. The apparatus according to claim 1 wherein said front side material is composed of one or more individual pieces.

3. The apparatus according to claim 1 wherein said chemically treated aromatically emissive substances are selected from the group consisting of potpourri, perfumed oils, natural perfumes, natural biological liquids, and combinations thereof.

4. The apparatus according to claim 1 wherein said material is selected from the group consisting of carpet, fake-fur, felt, filter base, woven cloth, and combinations thereof.

5. The apparatus according to claim 1 wherein said material is washable.

6. The apparatus according to claim 1 wherein said recognizable shape is selected from the group consisting of circles, squares, triangles, rectangles, ellipses, hearts, animal faces, animal bodies, birds, biological vegetation, and combinations thereof, and represents unartistic configurations.

7. The apparatus according to claim 1 wherein said means for attaching together said front side and rear side at overlying edges is selected from the group consisting of glue, thread, clips, fasteners, staples, tapes, Velcro-type tapes, and combinations thereof.

8. An apparatus for an air freshener comprising:
a body having a front side and a rear side composed of material, wherein said front side is further made into an identifiable shape, and wherein said material is selected from the group consisting of carpet, fake-fur, felt, filter base, loosely woven cloth, and combinations thereof, and further where said front side material allows air ventilation and is composed of one or more individual pieces;
means for attaching together said front side and rear side at overlying edges forming an inner chamber capable of holding chemically treated aromatically emissive substances, and further forming a charging slit opening into said chamber capable of positioning said chemically treated aromatically emissive substances; and
means for connecting said body to an external fixed location.

9. The apparatus according to claim 8 wherein said material is washable.

10. The apparatus according to claim 8 wherein said recognizable shape is selected from the group consisting of circles, squares, triangles, rectangles, ellipses, hearts, animal faces, animal bodies, birds, biological vegetation, and combinations thereof, and represents unartistic configurations.

11. The apparatus according to claim 8 wherein said means for connecting said body to an external fixed location is selected from the group consisting of hooks, strings, rubber bands, magnets, tapes, Velcro-type tapes, staples, clips, fasteners, and combinations thereof.

12. The apparatus according to claim 8 wherein said means for attaching together said front side and rear side at overlying edges is selected from the group consisting of glue, thread, clips, fasteners, staples, tapes, Velcro-type tapes, and combinations thereof.
